(19) United States
(12) Patent Application Publication
(10) Pub. No.: US 2022/0251153 A1
(43) Pub. Date: Aug. 11, 2022

(54) PEPTIDES FOR THE TREATMENT OF RENAL DISORDERS

(71) Applicants: THE GEORGE WASHINGTON UNIVERSITY, Washington, DC (US); RAMOT AT TEL-AVIV UNIVERSITY LTD., Tel-Aviv (IL)

(72) Inventors: Santiago CUEVAS GONZALEZ, Washington, DC (US); Pedro A. JOSE, Washington, DC (US); Daniel OFFEN, Tel-Aviv (IL); Rolondo EISEN, Panama City (PA)

(21) Appl. No.: 17/675,910

(22) Filed: Feb. 18, 2022

Related U.S. Application Data
(63) Continuation of application No. PCT/US20/48539, filed on Aug. 28, 2020.
(60) Provisional application No. 62/894,265, filed on Aug. 30, 2019.

(51) Int. Cl. C07K 14/47; A61P 13/12

(52) U.S. Cl. CPC C07K 14/4702; A61K 38/00; A61P 13/12

(57) ABSTRACT

Provided are methods for treating a renal disorder using DJ-1 related peptides and compositions thereof.

### CROSS REFERENCE TO RELATED APPLICATIONS

This application is a continuation of International Application No. PCT/US20/48539, filed Aug. 28, 2020, for PEPTIDES FOR THE TREATMENT OF RENAL DISORDERS, which claims the benefit of U.S. Provisional Application No. 62/894,265, filed on Aug. 30, 2019, the disclosures of which are hereby incorporated by reference in their entireties.

### GOVERNMENT LICENSE RIGHTS

The present disclosure was made with government support under P01 HL074940 and R01 DK039308 awarded by the National Institutes of Health. The government has certain rights in the invention.

### INCORPORATION OF SEQUENCE LISTING

A Sequence Listing is included herewith as a text file entitled “091019-663922_ST25.txt” created on or about Aug. 28, 2020 and having a size of 6.72 KB. The contents of the text file are incorporated by reference herein in their entirety.

### FIELD

Provided are methods for treating a renal disorder using DJ-1 related peptides and compositions thereof.

### BACKGROUND

Renal or kidney disorders involve an alteration in the normal physiology or function of the kidney. Renal disorders can result from a wide range of acute and chronic conditions and events including physical and biological injury or trauma and various inflammatory and autoimmune diseases. For example, diabetic nephropathy is the most common cause of renal failure worldwide. Regardless of the initial cause, some kidney disorders are characterized by progressive destruction of the renal parenchyma which triggers the collagen accumulation affecting the nephrons function. This progression often leads to chronic kidney disease (CKD) and end stage renal disease and failure (ESRD).

Persons with CKD have significantly higher rates of morbidity, mortality, hospitalizations and healthcare utilization. The prevalence of CKD stages has continued to increase as have the prevalence of diabetes and hypertension, which are respectively etiologic in CKD cases. However, the only current treatment of CKD and ESRD is renal replacement therapy via dialysis or kidney transplantation. There remains a need in the art to provide alternative therapies for treating kidney disorders.

### SUMMARY

The present disclosure is based, at least in part, on the unexpected discovery that that administration of a synthetic DJ-1 peptide successfully conferred protective effects on renal injury, renal fibrosis and renal inflammation associated with CKD in models of renal disease. These results suggest that certain peptides derived from DJ-1 (“DJ-1 peptides”) have a utility in the treatment of kidney disorders.

Accordingly, an aspect of the disclosure provide methods of treating a renal disorder in a subject in need of such treatment by administering to the subject an effective amount of an isolated peptide or peptide mimetic. In some embodiments, the isolated peptide or peptide mimetic thereof may be no longer than about 25 amino acids in length. In some embodiments, the isolated peptide or peptide mimetic thereof can be at least 5 to 20 consecutive amino acids from the amino acid sequence set forth as SEQ ID NO: 1. In some embodiments, the isolated peptide or peptide mimetic thereof can have an amino acid sequence of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, or SEQ ID No: 11. In specific examples, the isolated peptide or peptide mimetic thereof can be SEQ ID NO: 2.

In some embodiments, isolated peptide or peptide mimetic used in the methods disclosed herein can be attached to at least one cell penetrating agent. In some examples, a cell penetrating agent can have an amino sequence of SEQ ID NO: 12, SEQ ID NO: 13, or SEQ ID NO: 14.

In some embodiments, methods disclosed herein encompass administering an isolated peptide or peptide mimetic thereof having the amino acid sequence set forth in SEQ ID NO: 15.

In some other embodiments, methods disclosed herein can treat a renal disorder such as glomerulonephritis, acute kidney injury, polycystic kidney/renal disease, renal artery stenosis, lupus nephritis, diabetic nephropathy, interstitial nephritis, tubulo-interstitial nephritis, pyelonephritis, chronic kidney disease, focal segmental glomerulosclerosis, reflux nephropathy, and mixtures thereof.

In certain examples, methods disclosed herein can treat chronic kidney disease (CKD). In some aspects, methods disclosed herein can encompass administration of an isolated peptide or peptide mimetic thereof in an amount effective to slow or prevent the progression of chronic kidney disease. In some examples, an isolated peptide or peptide mimetic thereof disclosed herein can be administered in an amount effective to slow or prevent the progression of chronic kidney disease from stage I to stage II, from stage II to stage III, from stage III to stage IV, or from stage IV to stage V.

In certain examples, methods disclosed herein can treat a renal disorder wherein the renal disorder is diabetic nephropathy. In some aspects, methods disclosed herein can encompass administration of an isolated peptide or peptide mimetic thereof in an amount effective to slow or prevent the progression of diabetic nephropathy.

In certain examples, methods disclosed herein can treat a renal disorder wherein the renal disorder is renal fibrosis. In some aspects, methods disclosed herein can encompass administration of an isolated peptide or peptide mimetic thereof in an amount effective to slow or prevent the progression of renal fibrosis.

In some embodiments, methods disclosed herein can prevent renal fibrosis, renal inflammation and/or renal injury in a subject in need thereof.

In some embodiments, methods disclosed herein can further include administering to the subject an additional therapy. In some examples, that additional therapy can be selected from an anti-diabetic agent, a cytokine, a growth factor, an anti-inflammatory agent, an anti-coagulant agent, an agents that lowers or reduces blood pressure, an agent that reduces cholesterol, triglycerides, LDL, VLDL, or lipoprotein(a) or increases HDL, an agent that modulates the level of cholesterol-regulating proteins, and mixtures thereof.

In some embodiments, methods disclosed herein can treat a renal disorder in a cat. In other embodiments, methods disclosed herein can treat a renal disorder in a human. In some examples, methods disclosed herein can treat a renal disorder in a human with type 1 or type 2 diabetes. In some other examples, methods disclosed herein can treat a renal disorder in a human with type 1 or type 2 diabetes by co-administering to the human subject one or more anti-diabetic agents. In some aspects, the anti-diabetic agents that can be used in the methods disclosed herein can be selected from the group of sulfonylurea, glimepiride, glisentide, sulfonylurea, AY31637; biguanide, metformin, alpha-glucosidase inhibitor, acarbose, miglitol, thiazol-idinedione, troglitazone, pioglitazone, rosiglitazone, glipizide, balaglitazone, rivoglitazone, netoglitazone, troglitazone, englitazone, AD 5075, T 174, YM 268, R 102380, NC 2100, NIP 223, NIP 221, MK 0767, ciglitazone, adaglitazone, CLX 0921, darglitazone, CP 92768, BM 152054, a glucagon-like-peptide (GLP) or a GLP analog or agonist of GLP-1 receptor, insulin or analogues or mimetics thereof, and mixtures thereof.

In some embodiments, the present application provides methods for treating (e.g. preventing and/or slowing or delaying the rate of progression of) a kidney disorder in a subject in need thereof, comprising administering to the subject an effective amount of an isolated DJ-1 related peptide as herein described, or an effective amount of a pharmaceutical composition which includes an isolated DJ-1 related peptide and one or more pharmaceutically acceptable carriers. In an embodiment, a DJ-1 related peptide as herein described for use in the treatment of a kidney disorder is provided. In an embodiment, a unit dose formulation for treating a kidney disorder in subject comprising a DJ-1 related peptide as described herein and one or more pharmaceutically acceptable carriers is provided.

Another aspect of the present disclosure provides a composition, the composition encompassing a synthetic peptide having at least 20 consecutive amino acids from the amino acid sequence set forth as SEQ ID NO: 1, wherein the isolated peptide or peptide mimetic thereof is no longer than 25 amino acids in length. In some embodiments, a composition disclosed herein can further include at least one additional treatment agent. In some other embodiments, a composition disclosed herein can further include at least one pharmaceutically acceptable carrier and/or excipient. In some examples, a composition disclosed herein can further include at least one pharmaceutically acceptable carrier that is suitable for intravenous delivery.

In some embodiments, a synthetic peptide of the compositions disclosed herein can be coupled to at least one a kidney-specific targeting agent. In some examples, a kidney-specific targeting agent can be a peptide, an antibody, a compound, or a combination thereof.

In some embodiments, a synthetic peptide of the compositions disclosed herein can be packaged within a particle. In some examples, a particle disclosed herein can include at least one kidney-specific targeting agent conjugated to the particle surface. In some examples, a particle disclosed herein can include least one additional treatment agent packaged within the particle.

In some embodiments, compositions including at least one additional treatment agent as disclosed herein can include an additional treatment agent selected from an anti-diabetic agent, a cytokine, a growth factor, an anti-inflammatory agent, an anti-coagulant agent, an agents that lowers or reduces blood pressure, an agent that reduces cholesterol, triglycerides, LDL, VLDL, or lipoprotein(a) or increases HDL, or an agent that modulates the level of cholesterol-regulating proteins.

In some embodiments, compositions disclosed herein encompassing a synthetic peptide as disclosed herein can further include a macromolecular carrier. In some examples, a macromolecular carrier can be less than 30,000 Da. In some other examples, a macromolecular carrier can be an enzyme, an immune protein, or a peptide hormone.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIGS. 1A-1G show images depicting Sirius red staining of kidney sections harvested from C57Bl6 and DJ-1 knockout mice 14 days after which Unilateral Ureter Obstruction (UUO) was performed. FIGS. 1A-1C show Sirius red staining in C57Bl6 mice that were untreated (FIG. 1A), treated with scrambled peptide 24-hours prior to and for 14 days after UUO (FIG. 1B), and treated with ND-13 (a DJ-1 related peptide) 24-hours prior to and for 14 days after UUO (FIG. 1C). FIGS. 1D-1F show Sirius red staining in DJ-1 knockout mice that were untreated (FIG. 1D), treated with scrambled peptide 24-hours prior to and for 14 days after UUO (FIG. 1E), and treated with ND-13 24-hours prior to and for 14 days after UUO (FIG. 1F). FIG. 1G shows a graph depicting quantification of the amount of collagen in the kidneys of mice from each experimental group as determined by Sirius Red staining.

FIGS. 2A and 2B show graphs depicting urinary concertation of neutrophil gelatinase-associated (NGAL)—a marker of renal damage—as quantified by a commercial ELISA assay kit. *P<0.05, one-way ANOVA, Holm-Sidak test; n=4/5 mice per group. FIG. 2A shows a graph of NGAL measured from urine collected from C57Bl6 mice that were untreated, C57Bl6 mice treated with scrambled peptide 24-hours prior to and for 14 days after UUO, and C57Bl6 mice treated with ND-13 (a DJ-1 related peptide) 24-hours prior to and for 14 days after UUO. FIG. 2B shows a graph of NGAL measured from urine collected from DJ-1 knockout mice that were untreated, DJ-1 knockout mice treated with scrambled peptide 24-hours prior to and for 14 days after UUO, and DJ-1 knockout mice treated with ND-13 24-hours prior to and for 14 days after UUO.

FIGS. 3A-311 show graphs depicting the effect of ND-13 (a DJ-1 related peptide) on mRNA expression of inflammatory and fibrotic markers in mice with UUO. FIGS. 3A-3D are graphs showing the mRNA expression level of TNF-alpha (FIG. 3A), IL-6 (FIG. 3B), Col1a1 (FIG. 3C), and TGF-beta (FIG. 3D) in kidneys harvested from C57Bl6 mice that were untreated, C57Bl6 mice treated with scrambled peptide 24-hours prior to and for 14 days after UUO, and C57Bl6 mice treated with ND-13 24-hours prior to and for 14 days after UUO. FIGS. 3E-3H are graphs showing the mRNA expression level of TNF-alpha (FIG. 3E), IL-6 (FIG. 3F), Col1a1 (FIG. 3G), and TGF-beta (FIG. 3H) in kidneys harvested from DJ-1 knockout mice that were untreated, DJ-1 knockout mice treated with scrambled peptide 24-hours prior to and for 14 days after UUO, and DJ-1 knockout mice treated with ND-13 24-hours prior to and for 14 days after UUO. All data in FIGS. 3A-3H were normalized by GAPDH and expressed as mean±S.E. *P<0.05 vs. vehicle-treated cells, ANOVA, n=4/5 mice per group.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure. It should be understood at the outset that although illustrative implementations of one or more embodiments are illustrated below, the disclosed apparatus and methods may be implemented using any number of techniques. The disclosure should in no way be limited to the illustrative implementations, drawings, and techniques illustrated herein, but may be modified within the scope of the appended claims along with their full scope of equivalents.

Renal or kidney disorders are characterized by progressive destruction of the renal parenchyma which triggers the collagen accumulation affecting the nephrons function, ultimately altering the normal physiology or function of the kidney in a deleterious manner. This progression often leads to chronic kidney disease (CKD) and ultimately renal failure

CKD is characterized by progressive loss of kidney function. Increased albuminuria and gradual, progressive loss of renal function are primary manifestations in CKD. CKD patients experience over time an increase in albuminuria, proteinuria, serum creatinine, and renal histopathological lesions. The glomerular filtration rate (GFR) is considered the best overall index of kidney function in stable, non-hospitalized patients, with lower GFR corresponding to more severe CKD. The five stages of CKD are based on the estimated filtration rate (eGFR) which is primarily determined by serum creatinine. Persons with CKD have significantly higher rates of morbidity, mortality, hospitalizations and healthcare utilization. The prevalence of CKD stages II-V has continued to increase as have the prevalence of diabetes and hypertension, which are respectively etiologic in approximately 40% and 25% of CKD cases. Diabetic nephropathy is the most common cause of renal failure worldwide. Renal oxidative stress and inflammation are two of the most important factors involved in the pathogenesis of diabetic nephropathy (Giacco and Brownlee, Circ Res., 29:107(9):1058-70 (2010)). The development of renal disease in diabetes patients, or other renal or kidney disorders, cannot be prevented with current pharmacological therapies. The present disclosure aims, in part, at developing treatment of renal disorders by targeting the oxidative stress underlying acceleration of renal injury progression.

DJ-1 (also known as Park 7) is a small 189 amino acid protein that is ubiquitously expressed and highly conserved among diverse species. DJ-1 was initially identified as an autosomal recessive gene associated with Parkinson's disease and is expressed in brain, heart, kidney, liver, pancreas, and skeleton muscle in humans and rodents (Nagakubo, et al., Biochem Biophys Res Commun., 13; 231(2):509-13 (1997)). DJ-1 is a multifunctional oxidative stress-response protein that functions as a redox-sensitive chaperone with intrinsic antioxidant properties, especially in mitochondria, and regulates the expression of several antioxidant genes (Liu et al., J. Neurochem., 1; 105(6):2435-53 (2008); Zhou and Freed, J Biol Chem., 30; 280(52):43150-8 (2005)). Another antioxidant-defense role of DJ-1 is to act as a stabilizer of Nrf2. Nrf2 (nuclear factor erythroid 2-related factor 2) is a transcription factor that regulates the expression of several antioxidant genes. Nrf2 attenuates the NFkappaB-inflammatory response, and suppresses macrophage inflammatory responses by blocking pro-inflammatory cytokine transcription (Li et al., Biochem Pharmacol., 76(11):1485-9 (2008); Kobayashi et al., Nat Commun., 23; 7:11624 (2016)). Nrf2 can inhibit the development and progression of several diseases affecting the kidney (Shelton et al., Kidney Int., 84(6):1090-5 (2013)). The present disclosure reports that DJ-1 can inhibit renal reactive oxygen species (ROS) production, at least in part, via activation of Nrf2-antioxidant genes.

The present disclosure aims at developing treatment of renal disorders, such as but not limited to diabetic nephropathy, with DJ-1 peptides administered in amounts effective to slow or prevent the progression of chronic kidney disease.

The present disclosure reports that administration of a synthetic DJ-1 related peptide successfully conferred protective effects on the renal injury, renal fibrosis and renal inflammation associated with CKD in models of renal disease.

Accordingly, provided herein are methods for slowing or preventing the progression of chronic kidney disease using a DJ-1 related peptide. Also provided herein are pharmaceutical compositions that encompass a DJ-1 related peptide and methods of formulating such compositions.

### I. Methods

In some aspects, the present disclosure provides methods for conferring protective effects on renal injury, renal fibrosis, renal inflammation, or a combination thereof in a subject in need by treatment with a synthetic DJ-1 peptide. In several embodiments, a method for treating a renal disorder in a subject in need thereof is provided and can include administering to the human or animal an effective amount of a DJ-1 related peptide.

A suitable subject includes a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In certain embodiments, the animal is a cat. In preferred embodiments, the subject is a human.

In various embodiments, a subject in need may have been diagnosed with a renal or kidney disease. In some aspects, a subject in need may have been diagnosed with disease known to cause an underlying a renal or kidney disease. In some aspects, the subject may have CKD. In some aspects, the subject may have diabetic kidney disease (e.g., diabetic nephropathy). In other aspects, a subject may have end stage renal failure. In yet other aspects, a subject may have diabetes-induced end stage renal failure.

In some embodiments, a subject may at least one symptom of kidney disease. In some aspects, a symptom of kidney disease can be proteinuria. In yet other aspects, a symptom of kidney disease can be renal fibrosis. In other aspects, a symptom of kidney disease can be at least about a 25% decrease in estimated glomerular filtration rate (eGFR) compared to eGFR of a subject without a kidney disease. In still other aspects, a symptom of kidney disease can be at least about a 25% decrease in urinary creatinine clearance compared to urinary creatinine clearance of a subject without a kidney disease. In yet other aspects, a symptom of kidney disease can be at least about a 25% decrease in renal blood flow compared to renal blood flow of a subject without a kidney disease. In still other aspects, a symptom of kidney disease can be at least about a 25% decrease in the concentration of a urinary marker of renal damage (e.g., NGAL) compared to the concentration of a urinary marker of renal damage in a subject without a kidney disease. In some other aspects, a symptom of kidney disease can be advanced CKD. In yet some other aspects, a symptom of kidney disease can be renal failure.

As used herein, CKD can encompass stages I to stage V of kidney failure. As used herein, a subjecting having Stage I CKD has mild kidney damage and an eGFR of 90 or greater. As used herein, a subjecting having Stage II CKD has mild kidney damage and an eGFR between 60 and 89. As used herein, a subjecting having Stage III CKD has moderate kidney damage and an eGFR between 30 and 59. As used herein, a subjecting having Stage IV CKD has moderate to severe kidney damage and an eGFR between 15 and 29. As used herein, a subjecting having Stage V CKD has an eGFR less than 15 and is very close to kidney failure or kidneys have completely failed.

As used herein, the term “treating” refers to the application or administration of a composition including a DJ-1 peptide and, optionally, one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

In embodiments used herein, the term “treat” or any variation thereof (e.g., treatment, treating, etc.), refers to any treatment of a subject identified as at risk for, or diagnosed with, a biological condition, such as “hypertension, diabetes, congestive heart failure, lupus, sickle cell anemia and various inflammatory, infectious and autoimmune diseases, kidney transplant, nephropathy (e.g. diabetic nephropathy or reflux nephropathy); CKD; glomerulonephritis; inherited diseases such as polycystic kidney disease; nephromegaly (extreme hypertrophy of one or both kidneys); nephrotic syndrome; ESRD; acute and chronic renal failure; interstitial disease; nephritis (e.g. lupus nephritis, interstitial nephritis, tubule-interstitial nephritis, or pyelonephritis); sclerosis, an induration or hardening of tissues and/or vessels resulting from causes that include, for example, inflammation due to disease or injury; renal fibrosis and scarring; renal-associated proliferative disorders; and other primary or secondary nephrogenic conditions, and fibrosis associated with dialysis following kidney failure. The term treat, as used herein, includes: (i) preventing or delaying the presentation of symptoms associated with the biological condition of interest in an at-risk subject who has yet to display symptoms associated with the biological condition (e.g., preventing the presentation of symptoms in a patient who is suffering from chronic kidney disease stages I-III, preventing organ transplant fibrosis, etc.); (ii) ameliorating the symptoms associated with the biological condition of interest in a patient diagnosed with the biological condition (e.g., eliminating fluid accumulation in a patient suffering from chronic kidney disease); (iii) preventing, delaying, or ameliorating the presentation of symptoms associated with complications, conditions, or diseases associated with the biological condition of interest in either an at-risk subject or a subject diagnosed with the biological condition; (iv) slowing, delaying or halting the progression of the biological condition (e.g., slowing, delaying or halting the progression of chronic kidney disease from Stage I to Stage II, Stage II to Stage III, Stage III to Stage IV, or Stage IV to Stage V; delaying, slowing or halting the progression of liver fibrosis to cirrhosis; etc.); (v) preventing, delaying, slowing, halting or ameliorating the cellular events of fibrosis (e.g., preventing, delaying, slowing, halting or ameliorating increased matrix production, inhibition of matrix degradation, modulation of matrix receptors to facilitate cell-matrix interactions, fibroblast activation, epithelial-to-mesenchymal transition, monocytic and lymphocytic cell infiltration, and/or cell apoptosis); (vi) reducing interstitial disease score; (vii) preventing, delaying, ameliorating, slowing, halting or reducing renal inflammation; (viii) preventing, delaying, ameliorating, slowing, halting or reducing renal injury (e.g. as measured by one or more markers of renal injury) (ix) preventing, slowing, halting or delaying the development of chronic kidney disease in at risk patients; and/or (x) augmenting patient renal activity (e.g., enhancing glomerular filtration rate).

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The terms “inhibiting,” “reducing,” or “prevention,” or any variation of these terms, when used in the claims and/or the specification includes any measurable decrease or complete inhibition to achieve a desired result.

The term “symptom(s)” as used herein, refers to common signs or indications that a subject is suffering from a specific condition or disease. As a non-limiting example, chronic kidney disease-related symptoms contemplated herein include, but are not limited to, reduced glomerular filtration rate; kidney damage; presence of protein, red and white blood cells, bacteria, crystals and/or casts in urine; accumulation of interstitial macrophages, ECM accumulation; loss of nephrons; need to urinate frequently; increased water retention (puffiness or swelling) in the legs, around the eyes, or in other parts of the body; high blood pressure; anemia; loss of appetite, nausea and vomiting; itching; easy bruising; pale skin; shortness of breath from fluid accumulation in the lungs; headaches; peripheral neuropathy; altered mental status (encephalopathy from the accumulation of waste products or uremic poisons); chest pain due to pericarditis; bleeding (due to poor blood clotting); bone pain and fractures; and abnormalities in kidney size. As another non-limiting example, diabetic nephropathy symptoms include, but are not limited to, protein in the urine, reduced glomerular filtration rate, peripheral edema, and raised arterial blood pressure.

The term “renal disorder” or “kidney disorder” means any renal disorder, renal disease, or kidney disease where there is any alteration in normal physiology and function of the kidney. This can result from a wide range of acute and chronic conditions and events, including, but not limited to, physical, chemical or biological injury, insult, trauma or disease, such as for example hypertension, diabetes, congestive heart failure, lupus, sickle cell anemia and various inflammatory, infectious and autoimmune diseases, HIV (or related diseases)-associated nephropathies (e.g., HIV-associated nephropathy (HIVAN)), coronaviruses (e.g., SARS-CoV, HCoV NL63, HCoV HKU1, MERS-CoV, SARS-CoV-2), RNA viruses, influenza viruses (e.g., influenzaviruses A, B, C, and D) and the like. This term includes but is not limited to diseases and conditions such as kidney transplant, nephropathy (e.g., diabetic nephropathy or reflux nephropathy); chronic kidney disease (CKD); glomerulonephritis; inherited diseases such as polycystic kidney disease (PKD); nephromegaly (extreme hypertrophy of one or both kidneys); nephrotic syndrome; end stage renal disease (ESRD); acute and chronic renal failure; interstitial disease; nephritis (e.g., lupus nephritis, systemic lupus erythematosus (SLE), interstitial nephritis, tubule-interstitial nephritis, or pyelonephritis); sclerosis, an induration or hardening of tissues and/or vessels resulting from causes that include, for example, inflammation due to disease or injury; renal fibrosis and scarring; renal-associated proliferative disorders; and other primary or secondary nephrogenic conditions. Fibrosis associated with dialysis following kidney failure and catheter placement, e.g., peritoneal and vascular access fibrosis is also included.

In some embodiments, the kidney disorder may be generally defined as a “nephropathy” or “nephropathies”. The terms “nephropathy” or “nephropathies” encompass all clinical-pathological changes in the kidney which may result in kidney fibrosis and/or glomerular diseases (e.g. glomerulosclerosis, glomerulonephritis) and/or chronic renal insufficiency, and can cause end stage renal disease and/or renal failure. In some embodiments, the terms “nephropathy” or “nephropathies” refers specifically to a disorder or disease where there is either the presence of proteins (i.e. proteinuria) in the urine of a subject and/or the presence of renal insufficiency. In certain preferred embodiments, the kidney disorder is diabetic nephropathy, a syndrome of albuminuria, declining glomerular filtration rate, artificial hypertension and increased cardiovascular risk in a patient with type 1 or type 2 diabetes.

The term “fibrosis” refers to abnormal processing of fibrous tissue, or fibroid or fibrous degeneration. Fibrosis can result from various injuries or diseases, and can often result from chronic transplant rejection relating to the transplantation of various organs. Fibrosis typically involves the abnormal production, accumulation, or deposition of extracellular matrix components, including overproduction and increased deposition of, for example, collagen and fibronectin. As used herein, the terms “kidney fibrosis” or “renal fibrosis” or “fibrosis of the kidney” refer to diseases or disorders associated with the overproduction or abnormal deposition of extracellular matrix components, particularly collagen, leading to the degradation or impairment of kidney function.

In some embodiments, a subject in need of treatment according to the methods described herein is a human or animal at risk of (i.e. susceptible to), or that has been diagnosed with, a kidney disorder. In various embodiments, the subject can be a feline or “cat”. In various embodiments, the subject can be a human patient. In some aspects, a human patient can be diagnosed as having or progressing toward stage I kidney disease, stage II kidney disease, stage III kidney disease, stage IV kidney disease or stage V kidney disease. In some embodiments, the methods and compositions described herein can be effective in halting or slowing the progression of the human patient's kidney disease (e.g. from stage I to stage II, from stage II to stage III, etc.). In other embodiments, the methods and compositions described herein can be effective in improving the human patient's kidney disease (e.g. from stage V to stage IV, from stage IV to stage III, etc.).

In some embodiments, the subject can be at risk or has been diagnosed with diabetes. In some embodiments, the subject can be a human patient with type 2 diabetes. In other embodiments, the subject can be a human patient with type 1 diabetes. In related embodiments, the subject can be a human patient with type 1 or type 2 diabetes, identified as at risk for, or diagnosed with, diabetic nephropathy. In some aspects, human patients with type 1 or type 2 diabetes at risk for diabetic nephropathy can include those with reduced glomerular filtration rate and/or those with microalbuminuria. In some examples, a human patient with microalbuminuria can have an urinary albumin excretion of about 30 mg/day to about 300 mg/day wherein “day” is equivalent to 24 hours.

In some embodiments, a DJ-1 related peptide can be administered with a second agent useful for treating a kidney disorder. In some aspects of this embodiment, a second agent may be other therapeutic agents, such as anti-diabetic agents, cytokines, growth factors, other anti-inflammatory agents, anti-coagulant agents, agents that will lower or reduce blood pressure, agents that will reduce cholesterol, triglycerides, LDL, VLDL, or lipoprotein(a) or increase HDL, agents that will increase or decrease levels of cholesterol-regulating proteins, anti-neoplastic drugs or molecules.

Exemplary second agents include, but are not limited to, agents used to treat diabetes, cyclophosphamide, either alone or in combination with mycophenolate mofetil (MMF) or prednisolone, or other corticosteroids, anti-inflammatory agents, azathioprine, IFN-gamma.

Exemplary anti-diabetic agents include, but are not limited to, 1) sulfonylureas (e.g., glimepiride, glisentide, sulfonylurea, AY31637); 2) biguanides (e.g., metformin); 3) alpha-glucosidase inhibitors (e.g., acarbose, miglitol); 4) thiazol-idinediones (e.g., troglitazone, pioglitazone, rosiglitazone, glipizide, balaglitazone, rivoglitazone, netoglitazone, troglitazone, englitazone, AD 5075, T 174, YM 268, R 102380, NC 2100, NIP 223, NIP 221, MK 0767, ciglitazone, adaglitazone, CLX 0921, darglitazone, CP 92768, BM 152054); 5) glucagon-like-peptides (GLP) and GLP analogs or agonists of GLP-1 receptor (e.g., exendin) or stabilizers thereof (e.g., DPP4 inhibitors, such as sitagliptin); and 6) insulin or analogues or mimetics thereof.

In any of the methods disclosed herein, an effective amount of the synthetic DJ-1 peptide can be given to a subject in need thereof to alleviate one or more symptoms associated with renal failure (e.g., CKD and CKD associated with diabetic nephropathy). “An effective amount” as used herein refers to a dose of a synthetic DJ-1 peptide which is sufficient to confer a therapeutic effect on a subject having or at risk of having renal failure. In some aspects, a therapeutic effect can be to slow the progression of renal disease, stop the progression of renal disease, and/or prevent the need for renal replacement therapy (e.g., dialysis and/or transplantation).

Effective amounts vary, as recognized by those skilled in the art, depending on route of administration, excipient usage, and co-usage with other active agents. Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Effective amounts can also vary, depending on rate of renal clearance and stage of kidney disease.

In some embodiments, a composition disclosed herein can be administered to a subject in need thereof once. In some embodiments, a composition disclosed herein may be administered to a subject in need thereof more than once. In other embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

In some instances, an initial dose of a synthetic DJ-1 peptide may be given to a subject for a first course of treatment (e.g., about a week to about a month), which may be followed by one or more maintenance doses. The concentration of synthetic DJ-1 peptide in the maintenance doses may be lower or higher than the initial dose in the first course of treatment. Alternatively or in addition, the interval between two consecutive maintenance doses may be longer than the interval between two initial doses in the first course of treatment. Length between doses and concentration of synthetic DJ-1 peptide may be adjusted according to factors within the knowledge and expertise of the health practitioner. In some examples, dosing regimens and dosing concentrations will depend on the stage of renal disease in the subject. In other examples, dosing regimens and dosing concentrations will depend on the subject's renal clearance rate. In other examples, dosing regimens and dosing concentrations will depend on whether or not the subject is on dialysis. In some aspects, dosing regimens and dosing concentrations will depend on whether or not the subject is on hemodialysis or peritoneal dialysis.

The number of times a composition may be administered to an subject in need thereof can depend on the discretion of a medical professional, the severity of the kidney disease, and the subject's response to the formulation. In some embodiments, a composition disclosed herein may be administered continuously; alternatively, the dose of drug being administered may be temporarily reduced or temporarily suspended for a certain length of time (i.e., a “drug holiday”). In some aspects, the length of the drug holiday can vary between 2 days and 1 year, including by way of example only, 2 days, 1 week, 1 month, 6 months, and 1 year. In another aspect, dose reduction during a drug holiday may be from 10%-100%, including by way of example only 10%, 25%, 50%, 75%, and 100%.

In various embodiments, the desired daily dose of compositions disclosed herein may be presented in a single dose or as divided doses administered simultaneously (or over a short period of time) or at appropriate intervals. In other embodiments, administration of a composition disclosed herein may be administered to a subject about once a day, about twice a day, about three times a day. In still other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition disclosed herein may be administered to a subject once about 4 weeks.

Conventional methods, known to those of ordinary skill in the art of medicine, can be used to administer a synthetic DJ-1 peptide-containing pharmaceutical composition to the subject in renal failure. For example, a synthetic DJ-1 peptide-containing pharmaceutical composition can be administered via, e.g., administered orally, parenterally, by inhalation spray, topically, rectally, nasally, buccally, vaginally or via an implanted reservoir.

In various embodiments, compositions disclosed herein may be administered by parenteral administration. As used herein, “by parenteral administration” refers to administration of the compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, intracardiac, intraarticular, or intracavernous injection. In other aspects, administration of the disclosed compositions by parenteral injection may be by slow or bolus methods as known in the field. In some embodiments, the route of administration by parenteral injection can be determined by the target location. In some aspects, compositions disclosed herein may be administered intrarenally. In other aspects, compositions disclosed herein may be administered under at least one kidney capsule of a subject in need thereof. In still other aspects, compositions disclosed herein may be administered by retrograde injection through the ureter.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water-soluble antibodies can be administered by the drip method, whereby a pharmaceutical formulation containing the compounds of Formula (I) and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the compounds of Formula (I), can be dissolved and administered in a pharmaceutical excipient such as Water-for-Injection, 0.9% saline, or 5% glucose solution.

For oral administration, a synthetic DJ-1 peptide-containing pharmaceutical composition can take the form of, for example, tablets or capsules, prepared by conventional means with acceptable excipients such as binding agents (for example, pre-gelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Also included are bars and other chewable formulations.

In some embodiments, a composition as disclosed may be initially administered followed by a subsequent administration of one for more different compositions or treatment regimens. In other embodiments, a composition as disclosed may be administered after administration of one for more different compositions or treatment regimens.

In some embodiments, the subject to be treated by the methods described herein may be a subject who has undergone or is subjecting to another therapy for a renal disease and/or a CKD-causing disease. The prior therapy may be complete. Alternatively, the therapy may be still ongoing. In other embodiments, the subject may be subject to a combined therapy involving the synthetic DJ-1 peptide disclosed herein and a second therapy for CKD. Exemplary therapies for CKD include, but are not limited to, mineralocorticoid-receptor antagonists, sodium/glucose cotransporter 2 inhibitors, anti-inflammatory drugs, and drugs that mitigate oxidative injury. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### II. Compositions

Aspects of the present disclosure include a peptide encompassing some part of the amino acid sequence for DJ-1 or a related sequence thereof. As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence. In other aspects of the present disclosure, a composition encompassing a DJ-1 peptide disclosed herein may further comprise at least one pharmaceutically acceptable excipient.

(a) DJ-1 Peptides

In various embodiments, compositions disclosed herein comprise a peptide encompassing some part of the amino acid sequence for DJ-1 or a related sequence thereof. In certain embodiments, a DJ-1 related peptide for use according to the methods herein described can be an isolated DJ-1 related peptide or peptide mimetic thereof, no longer than 25 amino acids in length and comprising at least 3, 4, 5, 6, 7, 8, 9, 10, 11, 12, 13, 14, 15, 16, 17, 18, 19 or 20 consecutive amino acids from the amino acid sequence set forth as SEQ ID NO: 1 (KGAEEMETVIPVDVMRRAGI). According to one embodiment of this aspect of the present disclosure, the peptide comprises no more than 10 consecutive amino acids or no more than 15 consecutive amino acids of SEQ ID NO: 1.

In related embodiments, a DJ-1 related peptide comprises or consist of a shorter In related embodiments, a DJ-1 related peptide comprises or consists of a shorter peptide derivative of the peptide having the amino acid sequence set forth as SEQ ID NO:1, including but not limited to SEQ ID NOs: 2-11 as provided in table 1 below.

TABLE 1

| SEQ ID NO | Amino Acid Sequence |
|---|---|
| 1 | KGAEEMETVIPVDVMRRAGI |
| 2 | KGAEEMETVIPVD |
| 3 | TVIPVDVMRRAGI |
| 4 | EMETVIPVDVMRR |
| 5 | KGAEEMETVIPVDVM |
| 6 | METVIPVDVMRRAGI |
| 7 | VDVMRRAGI |
| 8 | KGAEEMETVIPV |
| 9 | GAEEME |
| 10 | DVMRRAGI |
| 11 | TVIPV |
| 12 | VIP |

The term “peptide” as used herein refers to a polymer of natural or synthetic amino acids, encompassing native peptides (either degradation products, synthetically synthesized polypeptides or recombinant polypeptides) and peptidomimetics (typically, synthetically synthesized peptides), as well as peptoids and semipeptoids which are polypeptide analogs, which may have, for example, modifications rendering the peptides even more stable while in a body or more capable of penetrating into cells.

Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), a-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present disclosure may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 2 and 3 below list naturally occurring amino acids (Table 1) and non-conventional or modified amino acids (Table 2) which can be used with the present disclosure.

TABLE 2

| | Three Letter | One Letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 3

| Non-conventional amino acid | Code | Non-conventional amino acid | Code |
|---|---|---|---|
| α-aminobutyric acid | Abu | L-N-methylarginine | Nmarg |
| α-amino-α-methylbutyrate | Mgabu | L-N -methylasparagine | Nmasn |
| arninocyclopropane-carboxylate | Cpro | L-N-methylaspartic acid | Nmasp |
| aminoisobutyric acid | Aib | L-N -methylcysteine | Nmcys |
| arninonorbornyl-carboxylate | Norb | L-N-methylglutamine | Nmgin |
| cyclohexylalanine | Chexa | L-N-methylglutamic acid | Nmglu |
| cyclopentylalanine | Cpen | L-N -methylhistidine | Nmhis |
| D-alanine | Dal | L-N -methylisolleucine | Nmile |
| D-arginine | Darg | L-N-methylleucine | Nmleu |
| D-aspartic acid | Dasp | L-N-methyllysine | Nmlys |
| D-cysteine | Dcys | L-N-methylmethionine | Nmmet |
| D-glutamine | Dgln | L-N-methylnorleucine | Nmnle |
| D-glutamic acid | Dglu | L-N-me thy !norvaline | Nmnva |
| D-histidine | Dhis | L-N-methylornithine Nmorn | Nmorn |
| D-isoleucine | Dile | L-N-methylphenylalanine | Nmphe |
| D-leucine | Dleu | L-N-methylproline | Nmpro |
| D-lysine | Dlys | L-N-methylserine | Nmser |
| D-methionine | Dmet | L-N-methylthreonine | Nmthr |
| D-ornithine | Dorn | L-N-methyltryptophan | Nmtrp |
| D-phenylalanine | Dphe | L-N-methyltyrosine | Nmtyr |
| D-proline | Dpro | L-N-methylvaline | Nmval |
| D-serine | Dser | L-N-methylethylglycine | Nmetg |
| D-threonine | Dthr | L-N-methyl-t-butylglycine | Nmtbug |
| D-tryptophan | Dtrp | L-norleucine | Nle |
| D-tyrosine | Dtyr | L-norvaline | Nva |
| D-valine | Dval | α-methyl-aminoisobutyrate | Maib |
| D-α-methylalanine | Dmala | α-methyl-y-aminobutyrate | Mgabu |
| D-α-methylarginine | Dmarg | α ethylcyclohexylalanine | Mchexa |
| D-α-methylasparagine | Dmasn | α-methylcyclopentyl alanine | Mcpen |
| D-α-methylaspartate | Dmasp | α-methyl-aαnapthylalanine | Manap |
| D-α-methylcysteine | Dmcys | α-methylpenicillamine | Mpen |
| D-α-methylglutamine | Dmgln | N-(4-aminobutyl)glycine | Nglu |
| D-α-methylhistidine | Dmhis | N-(2-aminoethyl)glycine | Naeg |
| D-α-methylisoleucine | Dmile | N-(3-aminopropyl)glycine | Norn |
| D-α-methylleucine | Dmleu | N-amino-α-methylbutyrate | Nmaabu |
| D-α-methyllysine | Dmlys | α-napthylalanine | Anap |
| D-α-methylmethionine | Dmmet | N-benzylglycine | Nphe |
| D-α-methylornithine | Dmorn | N-(2-carbamylethyl)glycine | Ngln |
| D-α-methylphenylalanine | Dmphe | N-(carbamylmethyl)glycine | Nasn |
| D-α-methylproline | Dmpro | N-(2-carboxyethyl)glycine | Nglu |
| D-α-methylserine | Dmser | N-(carboxymethyl)glycine | Nasp |
| D-α-methylthreonine | Dmthr | N-cyclobutylglycine | Ncbut |
| D-α-methyltryptophan | Dmtrp | N-cycloheptylglycine | Nchep |
| D-α-methyltyrosine | Dmty | N-cyclohexylglycine | Nchex |
| D-α-methylvaline | Dmval | N-cyclodecylglycine | Ncdec |
| D-α-methylalnine | Dnmala | N-cyclododeclglycine | Ncdod |
| D-α-methylarginine | Dnmarg | N-cyclooctylglycine | Ncoct |
| D-α-methylasparagine | Dnmasn | N-cyclopropylglycine | Ncpro |
| D-α-methylasparatate | Dnmasp | N-cycloundecylglycine | Ncund |
| D-α-methylcysteine | Dnmcys | N-(2,2-diphenylethyl)glycine | Nbhm |
| D-N-methylleucine | Dnmleu | N-(3,3-diphenylpropyl)glycine | Nbhe |
| D-N-methyllysine | Dnmlys | N-(3-indolylyethyl) glycine | Nhtrp |
| N-methylcyclohexylalanine | Nmchexa | N-methyl-y-aminobutyrate | Nmgabu |
| D-N-methylornithine | Dnmorn | D-N-methylmethionine | Dnmmet |
| N-methylglycine | Nala | N-methylcyclopentylalanine | Nmcpen |
| N-methylaminosobutyrate | Nmaib | D-N-methylphenylalanine | Dnmphe |
| N-(1-methylpropyl)glycine | Nile | D-N-methylproline | Dnmpro |
| N-(2-methylpropyl)glycine | Nile | D-N-methylserine | Dnmser |
| N-(2-methylpropyl)glycine | Nleu | D-N-methylserine | Dnmser |
| D-N-methyltryptophan | Dnmtrp | D-N-methylthreonine | Dnmthr |
| D-N-methyltyrosine | Dnmtyr | N-(1-methylethyl)glycine | Nva |
| D-N-methylvaline | Dnmval | N-methyla-napthylalanine | Nmanap |
| γ-aminobutyric acid | Gabu | N-methylpenicillamine | Nmpen |
| L-t-butylglycine Ncys | Tbug | N-(p-hydroxyphenyl)glycine | Nhtyr |
| L-ethylglycine | Etg | N-(thiomethyl)glycine | Ncys |
| L-homophenylalanine | Hphe | penicillamine | Pen |
| L-α-methylarginine | Marg | L-α-methylalanine | Mala |
| L-α-methylaspartate | Masp | L-α-methylasparagine | Masn |
| L-α-methylcysteine | Mcys | L-α-methyl-t-butylglycine | Mtbug |
| L-α-thylglutamine | Mgln | L-methylethylglycine | Metg |
| L-α-methylhistidine | Mhis | L-α-methylglutamate | Mglu |
| L-α-methylisoleucine | Mile | L-α-methylhomo phenylalanine | Mhphe |
| D-N-methylglutamine | Dnmgln | N-(2-methylthioethyl)glycine | Nmet |
| D-N-methylglutamate | Dnmglu | N-(3-guanidinopropyl)glycine | Narg |
| D-N-methylhistidine | Dnmhis | N-(1-hydroxyethyl)glycine | Nthr |
| D-N-methylisoleucine | Dnmile | N-(hydroxyethyl)glycine | Nser |
| D-N-methylleucine | Dnmleu | N-(imidazolylethyl)glycine | Nhis |
| D-N-methyllysine | Dnmlys | N-(3-indolylyethyl)glycine | Nhtrp |
| N-methylcyclohexylalanine | Nmchexa | N-methyl-γ-aminobutyrate | Nmgabu |
| D-N-methylornithine | Dnmorn | D-N-methylmethionine | Dnmmet |
| N-methylglycine | Nala | N-methylcyclopentylalanine | Nmcpen |
| N-methylaminoisobutyrate | Nmaib | D-N-methylphenylalanine | Dnmphe |
| N-(1-methylpropyl)glycine | Nile | D-N-methylproline | Dnmpro |
| N-(2-methylpropyl)glycine | Nleu | D-N-methylserine | Dnmser |
| D-N-methyltryptophan)glycine | Dnmtrp | D-N-methylthreonine | Dnmthr |
| L-α-methylarginine | Marg | N-(1-methylethyl)glycine | Nval |
| D-N-methyltyrosine | Dnmtyr | L-α-methylasparagine | Masn |
| D-N-methylvaline | Dnmval | N-methyla-napthylalanine | Nmanap |
| γ-aminobutyric acid | Gabu | N-methylpenicillamine | Nmpen |
| L-t-butylglycine | Tbug | N-(p-hydroxyphenyl)glycine | Nhtyr |
| L-ethylglycine | Etg | N-(thiomethyl)glycine | Ncys |
| L-homophenylalanine | Hphe | penicillamine | Pen |
| L-α-methylarginine | Marg | L-α-methylalanine | Mala |
| L-α-methylaspartate | Masp | L-α-methylasparagine | Masn |
| L-α-methylcysteine | Mcys | L-α-methyl-t-butylglycine | Mtbug |
| L-α-methylglutamine | Mgln | L-methylethylglycine | Metg |
| L-α ethylhistidine | Mhis | L-α-methylglutamate | Mglu |
| L-α thylisoleucine | Mile | L-α-methylhomophenylalanine | Mhphe |
| L-α-methylleucine | Mleu | N-(2-methylthioethyl)glycine | Nmet |
| L-α-methylmethionine | Mmet | L-α-methyllysine | Mlys |
| L-α-methylnorvaline | Mnva | L-α-methylnorleucine | Mnle |
| L-α-methylphenylalanine | Mphe | L-α-methylomithine | Morn |
| L-α-methylserine | Mser | L-α-methylproline | Mpro |
| L-α ethylvaline | Mtrp | L-α-methylthreonine | Mthr |
| L-α-methylleucine | Mvalnbhm | L-α-methyltyrosine | Mtyr |
| N-(N-(2,2-diphenylethyl) | Nnbhm | L-N-methylhomophenylalanine | Nmhphe |
| carbamylmethyl-glycine | | | |
| 1-carboxy-1-(2,2-diphenyl | Nmbc | N-(N-(3,3-diphenylpropyl) | Nnbhe |
| hylamino)cyclopropane | | carbamylmethyl(1)glycine | |
| L-N-methylalanine | Nmala | | |

The amino acids of the peptides of the present disclosure may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the present disclosure replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH2)5-COOH]—CO— for aspartic acid. Those non-conservative substitutions which fall under the scope of the present disclosure are those which still constitute a peptide having anti-bacterial properties.

As mentioned, the N and C termini of the peptides of the present disclosure may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl-protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present disclosure is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO—and benzyl-O—CO—, (substituted benzyl)-O—OC—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH2, —NHR2 and —NR2R3) or ester (i.e. the hydroxyl group at the C-terminus is replaced with —OR2). R2 and R3 are independently an aliphatic, substituted aliphatic, benzyl, substituted benzyl, aryl or a substituted aryl group. In addition, taken together with the nitrogen atom, R2 and R3 can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH2, NHCH3, —N(CH3)2, —NH(ethyl), —N(ethyl)2, —N(methyl) (ethyl), —NH(benzyl), —N(C1-C4 alkyl)(benzyl), —NH(phenyl), —N(C1-C4 alkyl) (phenyl), —OCH3, —O-(ethyl), —O-(n-propyl), —O-(n-butyl), —O-(iso-propyl), —O-(sec-butyl), —O-(t-butyl), —O-benzyl and —O-phenyl.

In preferred aspects, DJ-1 peptides disclosed herein are synthetic peptides. As used herein, the term “synthetic peptide” refers to a peptide having a distinct amino acid sequence from those found in natural peptides and/or proteins. A synthetic protein is not a subsequence of a naturally occurring protein, either the wild-type (i.e., most abundant) or mutant versions thereof. For example, a “synthetic DJ-1 peptide” is not a subsequence of naturally occurring DJ-1. A “synthetic peptide,” as used herein, may be produced or synthesized by any suitable method (e.g., recombinant expression, chemical synthesis, enzymatic synthesis, etc.).

DJ-1 related peptides useful in the methods described herein may be attached (either covalently or non-covalently) to a penetrating agent.

As used herein the phrase “penetrating agent” refers to an agent which enhances translocation of any of the attached peptide across a cell membrane.

According to one embodiment, the penetrating agent is a peptide and is attached to the DJ-1 related peptide (either directly or non-directly) via a peptide bond.

Typically, peptide penetrating agents have an amino acid composition containing either a high relative abundance of positively charged amino acids such as lysine or arginine, or have sequences that contain an alternating pattern of polar/charged amino acids and non-polar, hydrophobic amino acids.

Examples of peptide penetrating agents include those set forth in SEQ ID NOs: 11-13. By way of non-limiting example, cell penetrating peptide (CPP) sequences may be used in order to enhance intracellular penetration. CPPs may include short and long versions of TAT (YGRKKRR—SEQ ID NO: 13 and YGRKKRRQRRR—SEQ ID NO: 14) and HD (RRQRR—SEQ ID NO: 15). However, the disclosure is not so limited, and any suitable penetrating agent may be used, as known by those of skill in the art.

According to a particular embodiment, the peptides of the present disclosure are no longer than 25 amino acids (this includes the DJ-1 related peptide together with any additional attached sequence, such as a cell penetrating peptide as described above).

DJ-1 related peptides useful in the methods herein described may also comprise non-amino acid moieties, such as for example, hydrophobic moieties (various linear, branched, cyclic, polycyclic or hetrocyclic hydrocarbons and hydrocarbon derivatives) attached to the peptides; non-peptide penetrating agents; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Attaching the amino acid sequence component of the peptides of the present disclosure to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide. The association may be by the entrapment of the amino acid sequence within the other component (liposome, micelle) or the impregnation of the amino acid sequence within a polymer to produce the final peptide.

Peptides useful in the methods herein described may be linear or cyclic (cyclization may improve stability). Cyclization may take place by any means known in the art. Where the compound is composed predominantly of amino acids, cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the peptide may also take place through non-amino acid organic moieties comprised in the peptide.

Peptides useful in the methods herein described can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Large scale peptide synthesis is described by Andersson Biopolymers 2000; 55(3):227-50.

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques may also be used to generate the peptides of the present present disclosure. To produce a DJ-1 related peptide using recombinant technology, a polynucleotide encoding the peptide of the present disclosure is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present disclosure in the host cells.

In addition to being synthesizable in host cells, peptides useful in the methods herein described can also be synthesized using in vitro expression systems. These methods are well known in the art and the components of the system are commercially available.

(b) Pharmaceutical Formulations and Treatment Regimens

The DJ-1 related peptides for use according to the methods herein described may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the DJ-1 related peptides accountable for the biological effect.

(i) Pharmaceutically Acceptable Carriers and Excipients

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

In various embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In various embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In various embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition.

In various embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In specific embodiments, suitable thickening agent may be carboxymethylcellulose.

In various embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the present disclosure. In preferred aspects, additives will be biocompatible, and will not be harsh, abrasive, or allergenic.

In various embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In various embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

(ii) Dosage Formulations

Suitable routes of administration may, for example, include oral, rectal, transmucosal, especially transnasal, intestinal or parenteral delivery, including intramuscular, subcutaneous and intramedullary injections as well as, intravenous, intraperitoneal, intranasal injections.

One may administer the pharmaceutical composition in a local or systemic manner, for example, via local injection of the pharmaceutical composition directly into a tissue region of a patient.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present disclosure are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present disclosure may also be formulated in rectal compositions such as suppositories or retention enemas, using, e.g., conventional suppository bases such as cocoa butter or other glycerides.

Pharmaceutical compositions suitable for use in context of the present disclosure include compositions wherein the active ingredients are contained in an amount effective to achieve the intended purpose. In some embodiments, a therapeutically effective amount means an amount of active ingredients (DJ-1 related/synthetic peptides) effective to prevent, slow, alleviate or ameliorate symptoms of a disorder (e.g., CKD or CKD-induced diabetic nephropathy) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to brain or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

(iii) Kidney-Specific Targeting Agents and Formulations

In some embodiments, a DJ-1 peptide disclosed herein can be coupled to a kidney-specific targeting agent. As used herein, “a kidney-specific targeting agent” refers to an agent that localizes delivery of the DJ-1 peptide to the kidney in increases the amount of DJ-1 peptide delivered to the kidney. In some aspects, a composition disclosed herein that includes a kidney-specific targeting agent increases the amount of DJ-1 peptide delivered to the kidney by at least 10% to 99% compared to a composition that does not include a kidney-specific targeting agent. In some other aspects, a composition disclosed herein that includes a kidney-specific targeting agent increases the amount of DJ-1 peptide delivered to the kidney by at least 10%, at least 25%, at least 50%, at least 75%, or at least 99% compared to a composition that does not include a kidney-specific targeting agent. In some other aspects, a composition disclosed herein that includes a kidney-specific targeting agent prevents DJ-1 peptide delivery to other tissues that are not kidney tissues.

In embodiments, a kidney-specific targeting agent is a peptide, an antibody, a compound, or a combination thereof. In some aspects, a kidney-specific targeting agent is a peptide. Non-limiting examples of peptides that can be used to target the DJ-1 peptide disclosed herein to the kidney include, but are not limited to PKNGSDP (SEQ ID NO: 16), DSHKDLK (SEQ ID NO: 17), CYFQNCPRG (SEQ ID NO: 18), CLPVASC (SEQ ID NO: 19), G3-C12 (ANTPCG-PYTHDCPVKR)(SEQ ID NO: 20), ELRGDMAAL (SEQ ID NO: 21), GVKGVQGTL (SEQ ID NO: 22), HGVRGNLIS (SEQ ID NO: 23), GVRGQLATP (SEQ ID NO: 24), GMRDHRMTI (SEQ ID NO: 25), ETMQRDVRA (SEQ ID NO: 26), YRDFRDIWA (SEQ ID NO: 27), SLRDRGFT (SEQ ID NO: 28), HLNMWRDGG (SEQ ID NO: 29), GGAIKDTQN (SEQ ID NO: 30), LTCQVGRVH (SEQ ID NO: 31), (KKEEE)₃K (SEQ ID NO: 32), and modifications thereof. In some aspects, a kidney-specific targeting agent is an antibody. Non-limiting examples of antibodies that can be used to target the DJ-1 peptide disclosed herein to the kidney include, but are not limited to Anti-MHC II, Anti-CR2-Fc, Anti-VCAM 1, Anti-E-selectin, Anti-α8 integrin, Anti-CD11b, Dal K29, Anti-CD163, and modifications thereof. In some aspects, a kidney-specific targeting agent is a compound. Non-limiting examples of compounds that can be used to target the DJ-1 peptide disclosed herein to the kidney include, but are not limited to poly(vinylpyrrolidone-co-dimethyl maleic acid) (PVD), chitosan, and modifications thereof.

In some embodiments, compositions described herein can encompass a particle containing at least one a DJ-1 peptide disclosed herein. In some aspects, a particle can be a liposome, a nanoparticle, a micelle, and the like. In some examples, compositions described herein can be liposomes containing the DJ-1 peptide disclosed herein which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. The term “liposome” as used herein refers to a composition comprising an outer lipid layer membrane (e.g., a single lipid bi-layer known as unilamellar liposomes or multiple lipid bi-layers known as multilamellar liposomes) surrounding an internal aqueous space. See, e.g., Cullis et al., Biochim. Biophys Acta, 559: 399-420 (1987). A unilamellar liposome generally has a diameter in the range of about 20 to about 400 nanometers (nm), about 50 to about 300 nm, about 300 to about 400 nm, or about 100 to about 200 nm. A multilamellar liposome usually has a diameter in the range of about one to about ten micrometers and may comprise anywhere from two to hundreds of concentric lipid bilayers alternating with layers of an aqueous phase. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. The DJ-1 peptides disclosed herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In some embodiments, compositions herein can encompass particles containing at least one a DJ-1 peptide disclosed herein in addition to another therapeutic agent. In some examples, the therapeutic agent can be an agent for treating a renal disease, for example, an agent for treatment of CKD, ESRD, diabetic nephropathy and the like. Examples include, but are not limited to, sulfonylurea, glimepiride, glisentide, sulfonylurea, AY31637; biguanide, metformin, alpha-glucosidase inhibitor, acarbose, miglitol, thiazol-idinedione, troglitazone, pioglitazone, rosiglitazone, glipizide, balaglitazone, rivoglitazone, netoglitazone, troglitazone, englitazone, AD 5075, T 174, YM 268, R 102380, NC 2100, NIP 223, NIP 221, MK 0767, ciglitazone, adaglitazone, CLX 0921, darglitazone, CP 92768, BM 152054, a glucagon-like-peptide (GLP) or a GLP analog or agonist of GLP-1 receptor, insulin or analogues or mimetics thereof, Endothelin Receptor A antagonists, SGLT2 inhibitors, Mineralocorticoid-receptor antagonists, Vascular adhesion protein 1 inhibitors, JAK1/2 inhibitors, CCR2 antagonists, phosphodiesterase inhibitors, NOX1/4 inhibitors, ASK1 inhibitors, integrin, ACE inhibitors, ARBs, anti-inflammatory drugs, anti-fibrotic drugs, Erythropoietin, Iron, Vitamin D, Phosphorus binders, B-complex Vitamin, folic acid, Vitamin D, and mixtures thereof.

In some aspects, one or more a kidney-specific targeting agent can be conjugated to a particle surface. A kidney-specific targeting agent disclosed herein can be conjugated to a particle surface using conventional methods known in the art. In some examples, a kidney-specific targeting agent can be conjugated directly to a particle surface. In other examples, a kidney-specific targeting agent can be may be attached to the particle via a linker, for example, a polyethylene glycol (PEG) linker. In some examples, the lipid is 1,2-Distearoyl-sn-glycero-3-phosphorylethanolamine (DSPE).

In some embodiments, compositions described herein can at least one a DJ-1 peptide disclosed herein and a macromolecular carrier. Macromolecular carriers are useful for targeting drugs to the kidney, in particular low molecular weight glomerular proteins which can selectively accumulate in the kidneys. In some aspects, a macromolecular carrier for use herein can be less than about 30,000 Da. In some other aspects, a macromolecular carrier for use herein can be about 1,000 Da to about 30,000 Da. In some other aspects, a macromolecular carrier for use herein can be about 1,000 Da, about 5,000 Da, about 10,000 Da, about 15,000 Da, about 20,000 Da, about 25,000 Da, or about 30,000 Da. In some examples, a macromolecular carrier can be a low-molecular-weight protein (LMWP). In some examples, a macromolecular carrier can be an enzyme, an immune protein, or a peptide hormone. Non-limiting examples of macromolecular carriers include, but are not limited to, lysozymes, light chain immunoglobulins, insulin, and the like.

At least one a DJ-1 peptide disclosed herein can be coupled to a macromolecular carrier using methods known in the art. In some examples, a DJ-1 peptide disclosed herein can be coupled directly to a macromolecular carrier. In other examples, a DJ-1 peptide disclosed herein can be coupled to a macromolecular carrier via a spacer. Non-limiting examples of such spacers include, but are not limited to, oligopeptides, (poly-)alpha-hydroxy acids, pH sensitive cis-aconityl spacers, and the like. In some aspects, a DJ-1 peptide disclosed herein can be released from a macromolecular carrier once the composition reaches the kidney. In some examples, a DJ-1 peptide disclosed herein can be released from a macromolecular carrier once the composition reaches the kidney by a enzymatic or chemical hydrolysis of the bond complexing the peptide to the macromolecular carrier.

(iv) Kits

Compositions of the present disclosure may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the present disclosure formulated in a compatible pharmaceutical carrier may also be prepared, placed in an appropriate container, and labeled for treatment of an indicated condition, as is further detailed above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

### Definitions

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used in this specification and claim(s), it is understood that the terms “DJ-1 related peptide” and “DJ-1 synthetic peptide” can refer to a DJ-1 peptide of the same amino acid sequence and may be interpreted interchangeably.

Other objects, features and advantages of the present disclosure will become apparent from the following detailed description. It should be understood, however, that the detailed description and the specific examples, while indicating specific embodiments of the present disclosure, are given by way of illustration only, since various changes and modifications within the spirit and scope of the present disclosure will become apparent to those skilled in the art from this detailed description.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are given for the purpose of illustrating various embodiments of the present disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the present disclosure. Changes therein and other uses which are encompassed within the spirit of the present disclosure as defined by the scope of the claims will occur to those skilled in the art.

### Example 1. Administration of a DJ-1 Peptide Confers Protective Effect on Renal Injury in a Mouse Model of Kidney Disease

The renal dopamine 2 receptor (D2R) has antioxidant and anti-inflammatory properties mediated at least in part by renal DJ-1 (Armando et al., Hypertension, 49(3):672-8 (2007); Cuevas et al., Hypertension, 59(2):446-52 (2012); Cuevas et al., Int J Mol Sci., 14(9):17553-72 (2013)). Renal-selective silencing of DJ-1 in mice, via renal sub-capsular infusion of DJ-1 siRNA, impairs D2R-mediated antioxidant response and increases blood pressure-associated decreased renal Nrf2 expression and activity (Cuevas et al., Hypertension, 65(6):1251-7 (2015)). In addition, mice with germline deletion of DJ-1 (DJ-1−/−) have high constitutive blood pressure and decreased expression and activity of Nrf2. As such, targeting DJ-1 could inhibit renal reactive oxygen species (ROS) production, at least in part, via activation of Nrf2-antioxidant genes, providing a novel therapeutic target. To assess this, a synthetic DJ-1 peptide was generated.

A 13 amino acid chain from DJ-1 (KGAEEMETVIPVD (SEQ ID NO: 2)) was fused to a 7 amino acid cell-penetrating peptide (YGRKKRR (SEQ ID NO: 13)). The resulting 20 amino acid peptide, referred to herein as “ND-13”, had the following sequence: YGRKKRRKGAEEMETVIPVD (SEQ ID NO: 33).

The effect of ND-13 on renal disease was then assessed. Unilateral ureter obstruction (UUO) was chosen as a model of renal disease to determine if ND-13 has a protective effect. The UUO model is an known model of human and animal kidney disease, which mimics the effects of chronic kidney disease and diabetes (i.e., diabetic nephropathy), due to its capability of generating a progressive renal (tubule interstitial) fibrosis and has advantages of good reproducibility, a short time course and the presence of a contralateral kidney as control.

C57Bl/6 control mice as well as DJ-1 knockout mice were subjected to UUO in order to examine mechanisms involved in renal tubule interstitial fibrosis in vivo and to determine if ND-13 has a protective effect against renal disease. DJ-1 knockout mice used herein were from the original F2 hybrid strain (129/SvXC57BL/6J) that contained the mutated DJ-1 allele (DJ-1−/−) backcrossed to wild-type C57BL/6J for >20 generations and genotyped. Prior to UUO, ND-13 peptide and scrambled control peptide (3 mg/kg diluted in 100 ml of saline) were injected subcutaneously to C57Bl/6 and DJ-1 knockout mice the day before performing the complete UUO. For UUO surgery, the procedure was performed as follows. Briefly, mice were anesthetized with isofluorane (3-5% for induction and 1-3% for maintenance) and divided into two experimental groups: the UUO group and the sham operation group. In the UUO group, mice were shaved on the left side of the abdomen, a vertical incision was made through the skin with a scalpel and the skin was retracted. A second incision was made through the peritoneum to expose the kidney. The left ureter was ligated twice 15 mm below the renal pelvis with surgical silk and the ureter was then severed between the two ligatures. Then, the ligated kidney was placed gently back into its correct anatomical position and sterile saline was added to replenish loss of fluid. The incisions were sutured and mice were individually caged. Buprenorphine was used as an analgesic. 24 hours after UUO surgery, the ND-13 peptide and scrambled control peptide (3 mg/kg diluted in 100 ml of saline) were injected subcutaneously to C57Bl/6 and DJ-1 knockout mice and then injected every day thereafter for 14 days following UUO. Untreated normal C57Bl/6 mice served as a control. After 14 days, urine was collected and the mice were sacrificed. Tissues, including the kidneys, were obtained for analyses.

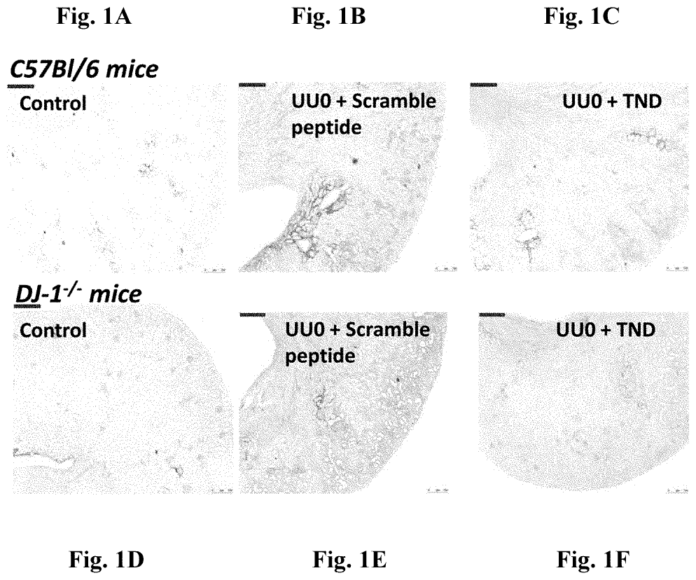

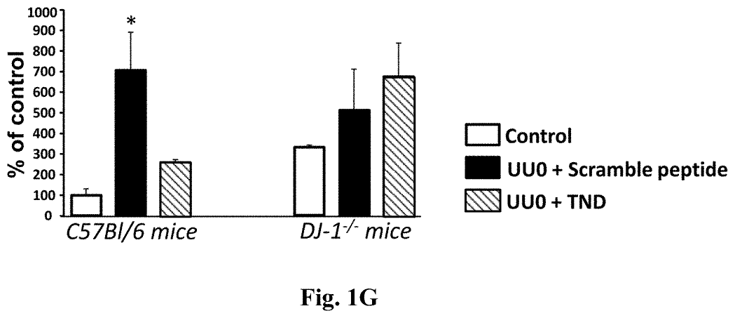

Renal interstitial fibrosis was assessed in the mice by Sirius Red staining. Briefly, thin sections (3 μm) of formalin-fixed paraffin-embedded mouse kidneys were deparaffinized in xylene and rehydrated with step-down concentrations of ethanol. The sections were rehydrated with tap water, stained with 0.1% Sirius Red F3BA solution in saturated aqueous picric acid overnight at room temperature. The sections were then washed in 0.01 N hydrochloric acid for 2 minutes, dehydrated in 3 changes of 100% ethanol, cleared in xylene and mounted on cover slips in a resinous medium. All image acquisition and morphometric analyses were performed under blinded conditions. Sirius Red staining was examined with an Olympus BX-41 microscope and the amount of collagen in the kidney was quantified by ImageJ software. Two images of about 70% of the renal cortical sections were used in the quantification. Sirius Red staining (FIGS. 1A-1G) showed that ND-13 treatment prevented fibrosis in the UUO model in C57Bl/6 mice but not in DJ-1 knockout mice. The data thus indicated utility of ND-13 in preventing connective tissue deposition—and resulting renal fibrosis—in the kidney parenchyma.

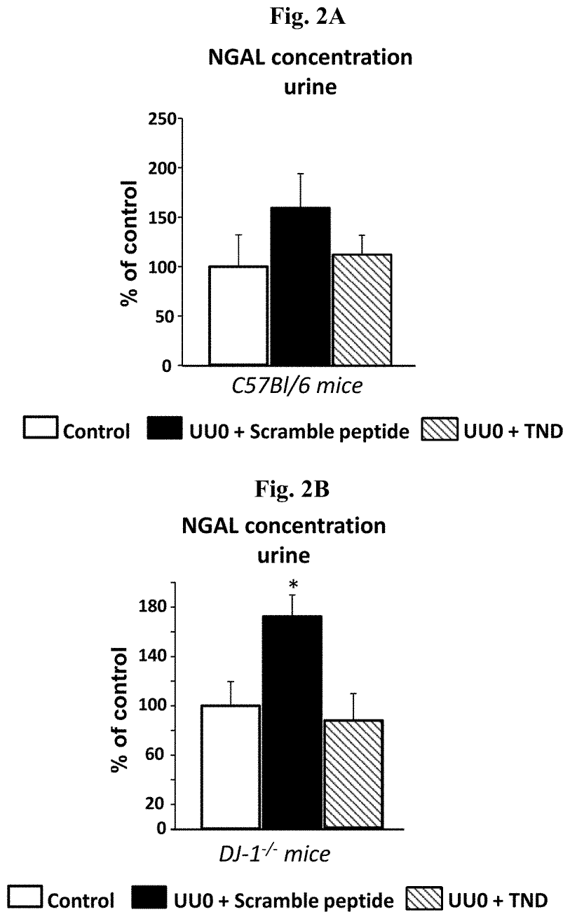

To further assess renal damage following UUO, urine was collected 14 days after UUO from ND-13-treated C57Bl/6 and DJ-1 knockout mice. Urine was subjected to an ELISA assay to measure urinary NGAL (neutrophil gelatinase-associated lipocalin) levels. FIGS. 2A-2B show that the amount of urinary NGAL, a marker of renal damage, was increased by UUO and that ND-13 treatment decreased urinary NGAL to control levels in C57Bl/6 (FIG. 2A) whereas DJ-1 knockout mice were not protected (FIG. 2B).

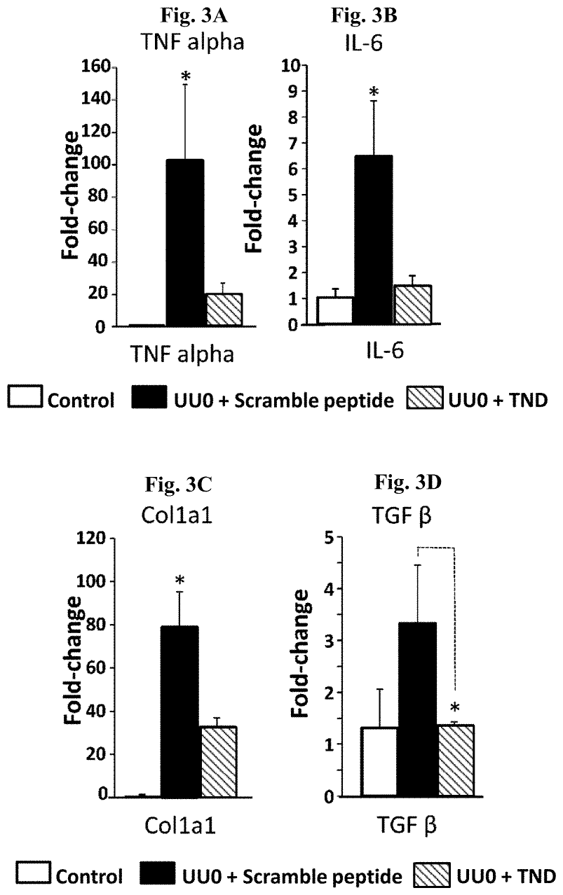

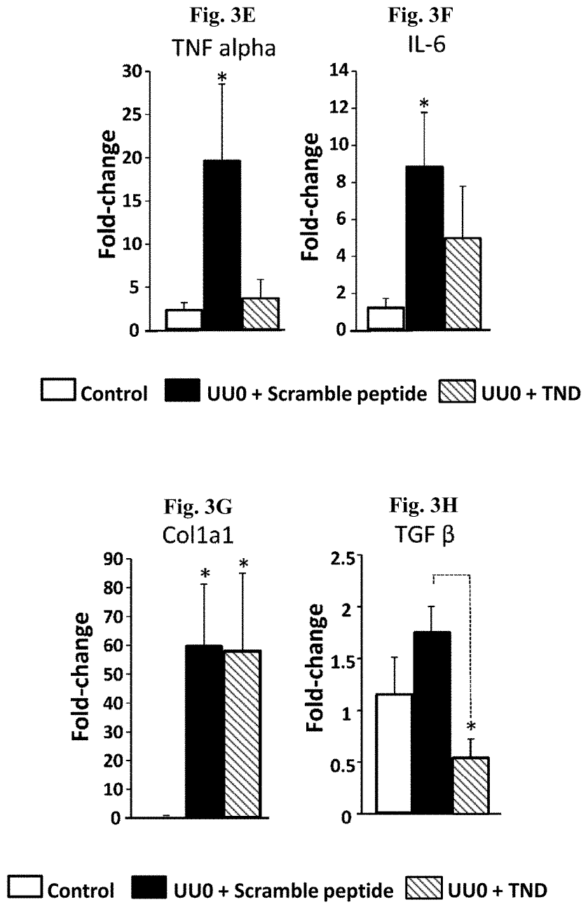

Next, the effect of ND-13 on mRNA expression of inflammatory and fibrotic markers in the kidneys of mice with UUO was tested. Briefly, RNA was extracted from whole renal tissue harvested from ND-13-treated C57Bl/6 and DJ-1 knockout mice 14 days after the UUO procedure. RNA extracted from untreated C57Bl/6 mice served as the control. The extracted RNA was subjected to qRT-PCR using standard methods. The mRNA expression of TNF-alpha (tumor necrosis factor alpha), IL-6 (Interleukin 6), Col1a1 (collagen, type I, alpha 1) and TGF-beta (transforming growth factor beta) were quantified by qRT-PCR in mice from each group. Data were normalized by expression of the house keeping gene GAPDH (glyceraldehyde 3-phosphate dehydrogenase). Data showed that UUO was associated with an increase in the renal mRNA expression of the inflammatory markers TNF-alpha and IL-6, the fibrosis marker Col1a1, and TGF-beta, which is an important factor associated with both renal inflammation and fibrosis (FIGS. 3A-311). The increased renal expressions of TNF alpha and IL-6, and TGF-beta associated with UUO was decreased by ND-13 in both C57Bl/6 mice (FIGS. 3A, 3B, and 3D) and DJ-1 knockout mice (FIGS. 3E, 3F, and 311). ND-13 also decreased the increased Col1a1 caused by UUO in C57Bl/6 (FIG. 3C) but not DJ-1 knockout mice (FIG. 3G). These data indicated that ND-13 confers protective effects on renal injury, renal fibrosis and renal inflammation—all of which are considered as crucial mechanisms in the pathogenesis of a variety of renal diseases such as glomerulonephritis, acute kidney injury (AKI), chronic kidney disease (CKD), polycystic kidney diseases (PKD), renal artery stenosis, lupus nephritis, and diabetic nephropathies, among others.

1. A method for treating a renal disorder in a subject in need of such treatment comprising administering to the subject an effective amount of an isolated peptide or peptide mimetic thereof, wherein the isolated peptide or peptide mimetic thereof is no longer than 25 amino acids in length and comprises at least 3 to 20 consecutive amino acids from an amino acid sequence set forth as SEQ ID NO: 1.

2. (canceled)

3. The method of claim 1, wherein the isolated peptide or peptide mimetic thereof comprises the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

4. (canceled)

5. The method of claim 1, wherein the isolated peptide or peptide mimetic thereof is attached to a cell penetrating agent.

6. The method of claim 5, wherein the isolated peptide or peptide mimetic thereof is attached to a peptide cell penetrating agent having an amino sequence selected from the group consisting of SEQ ID NO: 13, SEQ ID NO: 14, and SEQ ID NO: 15.

7. The method of claim 5, wherein the isolated peptide or peptide mimetic thereof attached to a peptide cell penetrating agent consists of the amino acid sequence set forth in SEQ ID NO: 33.

8. The method of claim 1, wherein the renal disorder is selected from the group consisting of glomerulonephritis, acute kidney injury, polycystic kidney/renal disease, renal artery stenosis, lupus nephritis, diabetic nephropathy, interstitial nephritis, tubulo-interstitial nephritis, pyelonephritis, chronic kidney disease, focal segmental glomerulosclerosis, reflux nephropathy, and any combination thereof.

9. (canceled)

10. The method of claim 1, wherein the isolated peptide or peptide mimetic thereof is administered in an amount effective to slow or prevent the progression of the renal disorder.

11-18. (canceled)

19. The method of claim 1, further comprising administering to the subject an additional therapy selected from an anti-diabetic agent, a cytokine, a growth factor, an anti-inflammatory agent, an anti-coagulant agent, an agents that lowers or reduces blood pressure, an agent that reduces cholesterol, triglycerides, LDL, VLDL, or lipoprotein(a) or increases HDL, an agent that modulates the level of cholesterol-regulating proteins, and mixtures thereof.

20. The method of claim 19, wherein the subject is a subject with type 1 or type 2 diabetes and the method comprises co-administering to the subject one or more anti-diabetic agents, selected from the group consisting of sulfonylurea, glimepiride, glisentide, sulfonylurea, AY31637; biguanide, metform in, alpha-glucosidase inhibitor, acarbose, miglitol, thiazol-idinedione, troglitazone, pioglitazone, rosiglitazone, glipizide, balaglitazone, rivoglitazone, netoglitazone, troglitazone, englitazone, AD 5075, T 174, YM 268, R 102380, NC 2100, NIP 223, NIP 221, MK 0767, ciglitazone, adaglitazone, CLX 0921, darglitazone, CP 92768, BM 152054, a glucagon-like-peptide (GLP) or a GLP analog or agonist of GLP-1 receptor, insulin or analogues or mimetics thereof, and mixtures thereof.

21-22. (canceled)

23. A composition, the composition comprising a synthetic peptide comprising at least 20 consecutive amino acids from the amino acid sequence set forth as SEQ ID NO: 1, wherein the synthetic peptide is no longer than 25 amino acids in length and the synthetic peptide is coupled to a kidney-specific targeting agent.

24. (canceled)

25. The composition of claim 23, wherein the kidney-specific targeting agent is a peptide, an antibody, a compound, or a combination thereof.

26. (canceled)

27. The composition of claim 25, wherein the kidney-specific targeting agent is a peptide selected from the group consisting of peptides having amino acid sequences as set forth in SEQ ID NOs: 16-32.

28. (canceled)

29. The composition of claim 25, wherein the kidney-specific targeting agent is an antibody selected from the group consisting of Anti-MHC II, Anti-CR2-Fc, Anti-VCAM 1, Anti-E-selectin, Anti-α8 integrin, Anti-CD11b, Dal K29, and Anti-CD163.

30. (canceled)

31. The composition of claim 25, wherein the kidney-specific targeting agent is a compound selected from the group consisting of poly(vinylpyrrolidone-co-dimethyl maleic acid) (PVD) and chitosan.

32-40. (canceled)

41. The composition of claim 23 further comprising at least one additional treatment agent selected from an anti-diabetic agent, a cytokine, a growth factor, an anti-inflammatory agent, an anti-coagulant agent, an agents that lowers or reduces blood pressure, an agent that reduces cholesterol, triglycerides, LDL, VLDL, or lipoprotein(a) or increases HDL, or an agent that modulates the level of cholesterol-regulating proteins.

42. The composition of claim 41, wherein at least one additional treatment agent is packaged within a particle.

43. The composition of claim 23, further comprising a macromolecular carrier.

44. The composition of claim 43, wherein the macromolecular carrier is less than 30,000 Da.

45. The composition of claim 43, wherein the macromolecular carrier is an enzyme, an immune protein, or a peptide hormone.

46. The composition of claim 23, further comprising a carrier suitable for intravenous delivery.

47-48. (canceled)


SEQUENCE LISTING (33 sequences)

SEQ ID NO: 1 - protein, 20 aa, Artificial Sequence
  Synthetic Sequence
KGAEEMETVIPVDVMRRAGI

SEQ ID NO: 2 - protein, 13 aa, Artificial Sequence
  Synthetic Sequence
KGAEEMETVIPVD

SEQ ID NO: 3 - protein, 13 aa, Artificial Sequence
  Synthetic Sequence
TVIPVDVMRRAGI

SEQ ID NO: 4 - protein, 13 aa, Artificial Sequence
  Synthetic Sequence
EMETVIPVDVMRR

SEQ ID NO: 5 - protein, 15 aa, Artificial Sequence
  Synthetic Sequence
KGAEEMETVIPVDVM

SEQ ID NO: 6 - protein, 15 aa, Artificial Sequence
  Synthetic Sequence
METVIPVDVMRRAGI

SEQ ID NO: 7 - protein, 9 aa, Artificial Sequence
  Synthetic Sequence
VDVMRRAGI

SEQ ID NO: 8 - protein, 12 aa, Artificial Sequence
  Synthetic Sequence
KGAEEMETVIPV

SEQ ID NO: 9 - protein, 6 aa, Artificial Sequence
  Synthetic Sequence
GAEEME

SEQ ID NO: 10 - protein, 8 aa, Artificial Sequence
  Synthetic Sequence
DVMRRAGI

SEQ ID NO: 11 - protein, 5 aa, Artificial Sequence
  Synthetic Sequence
TVIPV

SEQ ID NO: 12 - protein, 3 aa, Artificial Sequence
  Synthetic Sequence
VIP

SEQ ID NO: 13 - protein, 7 aa, Artificial Sequence
  Synthetic Sequence
YGRKKRR

SEQ ID NO: 14 - protein, 11 aa, Artificial Sequence
  Synthetic Sequence
YGRKKRRQRRR

SEQ ID NO: 15 - protein, 5 aa, Artificial Sequence
  Synthetic Sequence
RRQRR

SEQ ID NO: 16 - protein, 7 aa, Artificial Sequence
  Synthetic Sequence
PKNGSDP

SEQ ID NO: 17 - protein, 7 aa, Artificial Sequence
  Synthetic Sequence
DSHKDLK

SEQ ID NO: 18 - protein, 9 aa, Artificial Sequence
  Synthetic Sequence
CYFQNCPRG

SEQ ID NO: 19 - protein, 7 aa, Artificial Sequence
  Synthetic Sequence
CLPVASC

SEQ ID NO: 20 - protein, 16 aa, Artificial Sequence
  Synthetic Sequence
ANTPCGPYTHDCPVKR

SEQ ID NO: 21 - protein, 9 aa, Artificial Sequence
  Synthetic Sequence
ELRGDMAAL

SEQ ID NO: 22 - protein, 9 aa, Artificial Sequence
  Synthetic Sequence
GVKGVQGTL

SEQ ID NO: 23 - protein, 9 aa, Artificial Sequence
  Synthetic Sequence
HGVRGNLIS

SEQ ID NO: 24 - protein, 9 aa, Artificial Sequence
  Synthetic Sequence
GVRGQLATP

SEQ ID NO: 25 - protein, 9 aa, Artificial Sequence
  Synthetic Sequence
GMRDHRMTI

SEQ ID NO: 26 - protein, 9 aa, Artificial Sequence
  Synthetic Sequence
ETMQRDVRA

SEQ ID NO: 27 - protein, 9 aa, Artificial Sequence
  Synthetic Sequence
YRDFRDIWA

SEQ ID NO: 28 - protein, 8 aa, Artificial Sequence
  Synthetic Sequence
SLRDRGFT

SEQ ID NO: 29 - protein, 9 aa, Artificial Sequence
  Synthetic Sequence
HLNMWRDGG

SEQ ID NO: 30 - protein, 9 aa, Artificial Sequence
  Synthetic Sequence
GGAIKDTQN

SEQ ID NO: 31 - protein, 9 aa, Artificial Sequence
  Synthetic Sequence
LTCQVGRVH

SEQ ID NO: 32 - protein, 16 aa, Artificial Sequence
  Synthetic Sequence
KKEEEKKEEEKKEEEK

SEQ ID NO: 33 - protein, 20 aa, Artificial Sequence
  Synthetic Sequence
YGRKKRRKGAEEMETVIPVD